(19) United States
(12) Patent Application Publication
(10) Pub. No.: US 2021/0299082 A1
(43) Pub. Date: Sep. 30, 2021

(54) FISETIN COMPOSITION AND A METHOD OF MAKING THE SAME

(71) Applicant: KYLE DANIEL BREWER, East Palo Alto, CA (US)

(72) Inventor: KYLE DANIEL BREWER, East Palo Alto, CA (US)

(21) Appl. No.: 17/211,823

(22) Filed: Mar. 25, 2021

Related U.S. Application Data
(60) Provisional application No. 63/001,083, filed on Mar. 27, 2020.

(51) Int. Cl. A61K 31/352; A61K 9/48; A61K 9/08; A61K 47/10

(52) U.S. Cl. CPC A61K 31/352; A61K 47/10; A61K 9/08; A61K 9/4808

(57) ABSTRACT

A fisetin composition is disclosed herein. The fisetin composition comprises fisetin and one or more elements so that the solubility of the fisetin is increased, especially in view of higher effectiveness, potency, and/or bioavailability in treatments. Apparatus for use with the fisetin composition are disclosed herein.

### PRIORITY

This US utility patent application claims priority from the provisional application Ser. No. 63/001,083 with the title A FISETIN COMPOSITION AND A METHOD OF MAKING THE SAME filed on 27 Mar. 2020, the subject matter of which is incorporated herein by its entirety.

### TECHNICAL FIELD OF THE INVENTION

This invention relates in general to the art of fisetin, and more particularly, to the art of fisetin composition with improved solubility, especially to benefit its effectiveness, potency, and/or bioavailability in treatments.

### BACKGROUND OF THE INVENTION

Neurodegeneration and cancer are diseases in need of treatments. Neurodegeneration treatments are especially needed, as the number of people being diagnosed with and dying from dementia is increasing. Molecules are being proposed as treatments that can alleviate the burden posed by neurodegeneration and cancer. Fisetin has shown particular promise as a treatment for neurodegeneration and has a good safety profile.

The major problem with fisetin is solubility. Fisetin has limited solubility in water and, therefore, may have limited absorption by patients if used in a treatment. Increasing solubility of fisetin can increase its bioavailability, potency, and/or effectiveness.

What is desired is to develop a way to increase solubility, bioavailability, potency, and/or effectiveness of fisetin.

### SUMMARY OF THE DISCLOSURE

A fisetin composition is provided, wherein the fisetin composition comprises: (a) fisetin; and (b) a second component selected from a group that comprises ethanol. Said group may further comprise oil, castor oil, coconut oil, sunflower oil, flaxseed oil, grape seed oil, sesame oil, avocado oil, almond oil, coconut oil, corn oil, cottonseed oil, rapeseed oil, canola oil, safflower oil, olive oil, palm oil, peanut oil, peppermint oil, sesame oil, hydrogenated soybean oil, soybean oil, medium chain triglyceride oil, long chain triglyceride oil, mineral oil, hydrogenated vegetable oil, vegetable oil, polyoxyl 35 castor oil, polyoxyl 40 hydrogenated castor oil, polyoxyl 60 hydrogenated castor oil, polyoxyl hydrogenated castor oil, polyethoxylated castor oil, and polyethylene glycol 40 hydrogenated castor oil. The group may further comprise lecithin, beeswax, wax, d-alpha-tocopherol, oleic acid, monoglyceride, diglyceride, triglyceride, water, alcohol, glycerin, polyethylene glycol, polyethylene glycol 400, polyethylene glycol 300, polyethylene glycol 1750, sodium dodecyl sulfate, sodium lauryl sulfate, polyethylene glycol 660 12-hydroxystearate, polyethylene glycol 15 hydroxystearate, polyethylene glycol 300 caprylic/capric glycerides, polyoxylglyceride, propanediol, glycine, polyoxyl 8 stearate, polyoxyl 40 stearate, triglyceride, tocopherol, sorbitan monooleate, sorbitan monostearate, sorbitan monolaurate, sorbitan monopalmitate, sorbitan, polyoxyl40 stearate, poloxamer, poloxamer 188, poloxamer 407, poloxamer, polyethylene oxide, polysorbate 20, polysorbate 80, polysorbate, polyethylene glycol glycerides, polyethylene glycol 300 linoleic glycerides, polyethylene glycol 300 oleic glycerides, polyethylene glycol 300 caprylic/capric glycerides, polyethylene glycol 400 linoleic glycerides, polyethylene glycol 400 oleic glycerides, polyethylene glycol 400 caprylic/capric glycerides, D-alpha-tocopherol polyethylene glycol 1000 succinate, dimethyl sulfoxide, lipid, phospholipid, a cocrystal, hydrogenated soy phosphatidylcholine, distearoylphosphatidylglycerol, L-alpha-dimyristoylphosphatidylcholine, L-alpha-dimyristoylphosphatidylglycerol, N-methyl-2-pyrrolidone, methanol, isopropanol, hard fat, fat, lotion, milk, cream, petroleum jelly, polyethylene glycol 35 castor oil, ethylene glycol, propylene glycol, diethylene glycol, cyclodextrin, alpha-cyclodextrin, beta-cyclodextrin, hydroxypropyl-beta-cyclodextrin, sulfobutylether-beta-cyclodextrin, soy fatty acid, vinegar, glycerol, diethyl ether, fruit essence, orange essence, lemon essence, pineapple essence, gelatin, gelatin hydrolysate, xylitol, sorbitol, polyethylene glycol 40 stearate, polyethylene glycol 8 caprylic/capric glyceride, mannitol, and cellulose. The group may further comprise nicotinamide riboside, nicotinamide mononucleotide, nicotinamide adenine dinucleotide, calcium, calcium carbonate, ascorbic acid, vitamin C, vitamin D, vitamin D1, vitamin D2, vitamin D3, vitamin D4, vitamin D5, vitamin E, vitamin K, vitamin K1, and vitamin K2. The group may further comprise Kolliphor, Kolliphor CS 12, Kolliphor CS 20, Kolliphor CS S, Kolliphor CS A, Kolliphor CS L, Kolliphor TPGS, Kolliphor SLS, Kolliphor SLS Fine, Kolliphor RH 40, Kolliphor PS 20, Kolliphor PS 60, Kolliphor PS 80, Kolliphor HS 15, Kolliphor EL, Kolliphor P 188, Kolliphor P 407, Kolliphor P 237, Kolliphor P 338, Kolliphor P 188 micro, Kolliphor P 407 micro, Kollisolv, Kollisolv MCT 70, Kollisolv CAP, Kollisolv GTA, Kollisolv PG, Kollisolv PEG 300, Kollisolv PEG 400, Kollisolv PYR, Kollisolv P 124, Kolliphor EL, Cremophor, Cremophor RH 40, Cremophor RH 410, Cremophor RH 60, Labrafil, Labrafil M-1944CS, Labrafil M-2125CS, Softigen 767, Labrasol, Gellucire, Gellucire 44/14, Solutol, and Solutol HS 15. It is noted that the second component can be any combinations of the compositions in the above groups.

The fisetin composition can be a pharmaceutical composition, medical composition, cosmetic composition, lotion composition, cream composition, scientific composition, research composition, dental composition, oral composition, veterinary composition, ophthalmologic composition, alcohol composition, beverage composition, sauce composition, food composition, injectable composition, intravenous composition, atomized composition, or sprayed composition, or any combinations thereof.

A kit capable of applying the above described fisetin compositions is provided.

### EXAMPLES OF THE INVENTION

In view of the foregoing, the present invention provides a fisetin composition with increased solubility, bioavailability, potency, and/or effectiveness of treatments for patients, wherein patients include, but are not limited to, humans, dogs, cats, horses, pets, and animals. The term treatment herein refers to both therapeutic treatment and prophylactic or preventative measures. The invention will be described in the following with selected examples. It will be appreciated by those skilled in the art that the following discussion is for demonstration purposes, and should not be interpreted as any limitations. In fact, any variations thereof are applicable.

In one embodiment of the invention, a fisetin composition comprises fisetin and a second component that is selected from a group that comprises ethanol. The group may further comprise water. In one example, the solubility of fisetin in water is substantially 0.05 mg/mL or more, such as 0.1 mg/mL or more, 0.15 mg/mL or more, or 0.2 mg/mL. In another example, the solubility of fisetin in ethanol is substantially 2 mg/mL or more, such as 4 mg/mL or more, 6 mg/mL or more, 8 mg/mL or more, or 10 mg/mL or more.

In yet another embodiment of the invention, the fisetin composition is used by a person. 500 mg of fisetin was consumed every day by a person for the period of 5 days. Also, 500 mg of fisetin combined with 40 mL of 40% ethanol and 60% water was consumed every day by a person for 5 days.

In still another embodiment of the invention, the fisetin composition is used to relieve joint pain in a person. A 34-year-old man ingested fisetin (500 mg) in capsule powder form for 5 consecutive days, fisetin (500 mg) mixed in 35 mL of MCT oil for 5 consecutive days, or fisetin (500 mg) mixed in 40 mL of 40% ethanol and 60% water for 5 consecutive days, each test separated by a month. Reported joint pain decreased only after consuming fisetin in 40% ethanol and 60% water, but not the other regimens. The subject also experienced a slight increase in metabolism, a slight increase in energy, and a slight weight decrease (approximately 1 kg or 2.2 lbs to a BMI 23.7).

In still another example of the embodiment, fisetin can be used to decrease blood glucose level. Fisetin (500 mg) was ingested by a 57-year-old man after mixing with 75 mL of 53% ethanol and 47% water for 3 consecutive days. After the administration, the subject's waking blood glucose level decreased from 5.7 mmol/L to 4.8 mmol/L. The subject also experienced a slight increase in metabolism, a slight increase in energy, a slight weight decrease (approx. 1 kg to a BMI 22.1), and slight muscle gain in a month afterwards without increased exercise.

In yet another example of the invention, fisetin can be used as a topical skin treatment. Fisetin was dissolved in polyethylene glycol containing titanium nanoparticles with a resulting fisetin concentration of 200 mM. Fisetin was applied to the skin of 20 people for 2 days. Subjects reported improved skin appearance after the trial.

In another example of the embodiment, the fisetin and the second component can be mixed in a container, which can be ready for use. In another example, the fisetin and second component can be placed in separate containers and mixed together later such as before use. This can be of great importance when components are in different storage conditions or when components are subject to shipping or travel limitations. This can be useful especially when fisetin and the second component are in different locations. In fact, components of the fisetin composition can be prepared and/or stored in multiple locations and/or placed in separate containers as needed. The components can be mixed together before use.

In another embodiment, the group of the second component may further comprise oil, castor oil, coconut oil, sunflower oil, flaxseed oil, grape seed oil, sesame oil, avocado oil, almond oil, coconut oil, corn oil, cottonseed oil, rapeseed oil, canola oil, safflower oil, olive oil, palm oil, peanut oil, peppermint oil, sesame oil, hydrogenated soybean oil, soybean oil, medium chain triglyceride oil, long chain triglyceride oil, mineral oil, hydrogenated vegetable oil, vegetable oil, polyoxyl 35 castor oil, polyoxyl 40 hydrogenated castor oil, polyoxyl 60 hydrogenated castor oil, polyoxyl hydrogenated castor oil, polyethoxylated castor oil, and polyethylene glycol 40 hydrogenated castor oil. The group may further comprise lecithin, beeswax, wax, d-alpha-tocopherol, oleic acid, monoglyceride, diglyceride, triglyceride, water, alcohol, glycerin, polyethylene glycol, polyethylene glycol 400, polyethylene glycol 300, polyethylene glycol 1750, sodium dodecyl sulfate, sodium lauryl sulfate, polyethylene glycol 660 12-hydroxystearate, polyethylene glycol 15 hydroxystearate, polyethylene glycol 300 caprylic/capric glycerides, polyoxylglyceride, propanediol, glycine, polyoxyl 8 stearate, polyoxyl 40 stearate, triglyceride, tocopherol, sorbitan monooleate, sorbitan monostearate, sorbitan monolaurate, sorbitan monopalmitate, sorbitan, polyoxyl40 stearate, poloxamer, poloxamer 188, poloxamer 407, poloxamer, polyethylene oxide, polysorbate 20, polysorbate 80, polysorbate, polyethylene glycol glycerides, polyethylene glycol 300 linoleic glycerides, polyethylene glycol 300 oleic glycerides, polyethylene glycol 300 caprylic/capric glycerides, polyethylene glycol 400 linoleic glycerides, polyethylene glycol 400 oleic glycerides, polyethylene glycol 400 caprylic/capric glycerides, D-alpha-tocopherol polyethylene glycol 1000 succinate, dimethyl sulfoxide, lipid, phospholipid, a cocrystal, hydrogenated soy phosphatidylcholine, distearoylphosphatidylglycerol, L-alpha-dimyristoylphosphatidylcholine, L-alpha-dimyristoylphosphatidylglycerol, N-methyl-2-pyrrolidone, methanol, isopropanol, hard fat, fat, lotion, milk, cream, petroleum jelly, polyethylene glycol 35 castor oil, ethylene glycol, propylene glycol, diethylene glycol, cyclodextrin, alpha-cyclodextrin, beta-cyclodextrin, hydroxypropyl-beta-cyclodextrin, sulfobutylether-beta-cyclodextrin, soy fatty acid, vinegar, glycerol, diethyl ether, fruit essence, orange essence, lemon essence, pineapple essence, gelatin, gelatin hydrolysate, xylitol, sorbitol, polyethylene glycol 40 stearate, polyethylene glycol 8 caprylic/capric glyceride, mannitol, and cellulose. The group may further comprise nicotinamide riboside, nicotinamide mononucleotide, nicotinamide adenine dinucleotide, calcium, calcium carbonate, ascorbic acid, vitamin C, vitamin D, vitamin D1, vitamin D2, vitamin D3, vitamin D4, vitamin D5, vitamin E, vitamin K, vitamin K1, and vitamin K2. The group may further comprise Kolliphor, Kolliphor CS 12, Kolliphor CS 20, Kolliphor CS S, Kolliphor CS A, Kolliphor CS L, Kolliphor TPGS, Kolliphor SLS, Kolliphor SLS Fine, Kolliphor RH 40, Kolliphor PS 20, Kolliphor PS 60, Kolliphor PS 80, Kolliphor HS 15, Kolliphor EL, Kolliphor P 188, Kolliphor P 407, Kolliphor P 237, Kolliphor P 338, Kolliphor P 188 micro, Kolliphor P 407 micro, Kollisolv, Kollisolv MCT 70, Kollisolv CAP, Kollisolv GTA, Kollisolv PG, Kollisolv PEG 300, Kollisolv PEG 400, Kollisolv PYR, Kollisolv P 124, Kolliphor EL, Cremophor, Cremophor RH 40, Cremophor RH 410, Cremophor RH 60, Labrafil, Labrafil M-1944CS, Labrafil M-2125CS, Softigen 767, Labrasol, Gellucire, Gellucire 44/14, Solutol, and Solutol HS 15. It is noted that the second component can be any combinations of the compositions in the above groups.

The fisetin composition can be a pharmaceutical composition, medical composition, cosmetic composition, lotion composition, cream composition, scientific composition, research composition, dental composition, oral composition, veterinary composition, ophthalmologic composition, alcohol composition, beverage composition, sauce composition, food composition, injectable composition, intravenous composition, atomized composition, or sprayed composition, or any combinations thereof.

Similar to the above examples, the fisetin and the second component can be prepared in different locations and/or stored in separate containers. The fisetin and second component can be mixed together to form the said fisetin composition before use, which will not be detailed herein for simplicity.

The fisetin composition as described above can be in any suitable forms especially for convenience of applications. For example, the fisetin can be a pharmaceutical composition, medical composition, cosmetic composition, lotion composition, cream composition, scientific composition, research composition, dental composition, oral composition, veterinary composition, ophthalmologic composition, alcohol composition, beverage composition, sauce composition, food composition, injectable composition, intravenous composition, atomized composition, or sprayed composition, or any combinations thereof.

In yet another example of the invention, a bottle, tube, or any other suitable containers containing fisetin can be provided. The fisetin is combined with the second component(s) selected from the group(s) detailed above.

In still yet another example, a fisetin kit is provided. The kit comprises a container that can be a bottle, a tube, or any other suitable containers. The container can be soft so that it is squeezable like a tube or a bottle with a hard body having a preferably safe opening. The container contains at least fisetin. A label or insert with instructions on how to use the fisetin in the container with an unprovided second component or unprovided second components selected from the group(s) detailed above is included in the kit.

In still yet another example, a plurality of containers are provided in a kit. At least one of the containers contains at least fisetin; and at least a second container contains a second component different from the said one of the containers. As an instance of the example, the first container may contain fisetin, wherein the second container contains the second component(s) selected from the group(s) detailed above. In another instance of the example, the first container may have fisetin and a first additional component, such as polyethylene glycol 400. The second container may have a second additional component such as ethanol.

The fisetin in the invention can be prepared in any ways. For example, the fisetin can be extracted from natural sources, including, but not limited to, Rhus succedanea, Cotinus coggygria, Gleditsia triacanthos, or Toxicodendron succedaneum.

In some applications, the fisetin can be present in a bottle, tube, or other suitable containers combined with corn oil, sunflower oil, olive oil, vinegar, glycerol, diethyl ether, propylene glycol, polyethylene glycol 300, polyethylene glycol 400, lecithin, ethanol, a combination of ethanol and water, a combination of lecithin and water, a combination of ethanol and lecithin, or a combination of ethanol and water and lecithin.

In some other examples, the fisetin can be present in a bottle, tube, or other suitable containers to be combined with lecithin later, or to be combined with ethanol and water later, or to be combined with ethanol and water and lecithin later or to be combined with water and lecithin later, or to be combined with lecithin and ethanol later.

In some yet other examples, the fisetin will be present in a bottle, tube, or other suitable containers to be combined with ethanol that is not provided, or to be combined with ethanol and water that is not provided, or to be combined with lecithin that is not provided, or to be combined with lecithin and ethanol that is not provided, or to be combined with lecithin and water that is not provided, or to be combined with lecithin and ethanol and water that is not provided.

In yet some other examples, the fisetin can be present in a tincture, such as an ethanol, vinegar, diethyl ether, glycerol, or propylene glycol tincture, or other suitable tinctures or variations thereof.

The fisetin composition can be useful in many applications. For example, the fisetin composition of this invention can be used as a treatment for psoriasis, neurodegeneration, Alzheimer's disease, Parkinson's disease, Huntinington's disease, amyotrophic lateral sclerosis, dementia, vascular dementia, brain fog, cancer, osteoarthritis, joint pain, aging, heart disease, high blood pressure, inflammation, inflammatory diseases, type 2 diabetes, insulin resistance, glucose intolerance, hypertension, high blood cholesterol, hyperinsulinemia, dyslipidemia, obesity, angina pectoris, stroke, respiratory problems, congestive heart failure, gallstones, fatty liver, gout, sleep apnea, reproductive health, fertility, erectile dysfunction, bladder control, psychological disorders, and depression, and many other diseases.

The fisetin composition can be applied in many suitable ways. For example, the fisetin composition of the invention can be administered orally. In other examples, the composition of the invention can be administered by local infusion or injection locally to the area in need of treatment. In still another embodiment, the composition of the invention can be administered by intravenous injection of the agent, optionally in saline.

The fisetin composition of this invention can be in any suitable forms such as: aqueous ethanol solution, lotions, creams, gels, multiple emulsions, suspensions, oils, balm, soaps, conditioner, shampoo, anhydrous compositions, powders, aqueous dispersions, sprays, emulsions, foams, aerosols, emulsions, hydroglycolic solution, hydrogels, dragees, syrups, jellies, gelatin coating poultices, slurries, clear slurry, ointment, mousse, hair cream, chewing gum, bars, tablets, pencils, capsules, gelatin capsules, soft capsules, hard capsules, granules, solutions, suspensions, emulsions, and polysaccharides films. In some other examples, the fisetin composition can be mixed with products including eye shadow, eye cream, washing foam, washing foam (cream), make-up remover, foundation, lip balm, lip gloss, lipstick, and powder.

Nanotechnology can be incorporated in the fisetin composition of this invention. In one example, nanoparticles can be provided to carry the fisetin composition. For example, fisetin composition can be coated on the exterior surfaces of nanoparticles. In some examples, the fisetin composition can be coated on a portion of a nanoparticle, whereas another portion of the nanoparticle surface can be coated by another material such as a bio-specific molecule or cluster. In some other examples, nanoparticles are porous nanoparticles, in which the fisetin composition can be embedded in the pores or vacancies or holes of the nanoparticles. In some other examples, fisetin and other compositions of the fisetin composition can be coated on different nanoparticles. For example, fisetin can be coated on a first group of nanoparticles; whereas another component of the fisetin composition such as ethanol or oil or vitamin can be coated on or carried by a second group of nanoparticles. In another example, a nanoparticle can be chemically bonded to fisetin, or a nanoparticle can be chemically bonded to fisetin through polyethylene glycol.

The nanoparticles can be composed of any suitable materials, such as ceramic or metallic materials. The nanoparticles can be further compromised of lecithin or lipid in the form of liposomes. Alternatively, the nanoparticles can be composed of molecules or the like. An advantage of using nanoparticles is to enhance the absorption of the fisetin solution. Of course, nanoparticles can provide many other benefits such as specificity and target orientated delivery. In some examples, using nanoparticles with the fisetin solution can avoid expected damage to the fisetin solution. For example wherein the solution comprises fisetin and ethanol and vitamin C, ethanol or vitamin C may be dissolved in the body not together with the fisetin. This problem can be solved by using proper nanoparticles.

In some of the examples of this invention, water is incorporated in the fisetin solution. Here, water can be any type of suitable water forms, such as distilled water, double distilled water, tap water, mineral water, sparkling water, spring water, milli-Q water, soda water, seltzer water, any other form of water, or any other form of drink.

The invention has been described in selected examples in the above. It will be appreciated by those skilled in the art that these examples are for demonstration purposes and should not be interpreted as limitations. Any variations are applicable.

We claim:

1. A fisetin composition comprising:
a first component selected from a first group that comprises fisetin; and
a second component selected from a second group that comprises ethanol.

2. The fisetin composition of claim 1, wherein the second group comprises water and ethanol.

3. The fisetin composition of claim 1 comprises fisetin, water, ethanol, and lecithin.

4. The fisetin composition of claim 1, wherein the second group further comprises: a composition of ethanol and water, oil, castor oil, coconut oil, sunflower oil, flaxseed oil, grape seed oil, sesame oil, avocado oil, almond oil, coconut oil, corn oil, cottonseed oil, rapeseed oil, canola oil, safflower oil, olive oil, palm oil, peanut oil, peppermint oil, sesame oil, hydrogenated soybean oil, soybean oil, medium chain triglyceride oil, long chain triglyceride oil, mineral oil, hydrogenated vegetable oil, vegetable oil, polyoxyl 35 castor oil, polyoxyl 40 hydrogenated castor oil, polyoxyl 60 hydrogenated castor oil, polyoxyl hydrogenated castor oil, polyethoxylated castor oil, and polyethylene glycol 40 hydrogenated castor oil. The group may further comprise lecithin, beeswax, wax, d-alpha-tocopherol, oleic acid, monoglyceride, diglyceride, triglyceride, water, ethanol, alcohol, glycerin, polyethylene glycol, polyethylene glycol 400, polyethylene glycol 300, polyethylene glycol 1750, sodium dodecyl sulfate, sodium lauryl sulfate, polyethylene glycol 660 12-hydroxystearate, polyethylene glycol 15 hydroxystearate, polyethylene glycol 300 caprylic/capric glycerides, polyoxylglyceride, propanediol, glycine, polyoxyl 8 stearate, polyoxyl 40 stearate, triglyceride, tocopherol, sorbitan monooleate, sorbitan monostearate, sorbitan monolaurate, sorbitan monopalmitate, sorbitan, polyoxyl40 stearate, poloxamer, poloxamer 188, poloxamer 407, poloxamer, polyethylene oxide, polysorbate 20, polysorbate 80, polysorbate, polyethylene glycol glycerides, polyethylene glycol 300 linoleic glycerides, polyethylene glycol 300 oleic glycerides, polyethylene glycol 300 caprylic/capric glycerides, polyethylene glycol 400 linoleic glycerides, polyethylene glycol 400 oleic glycerides, polyethylene glycol 400 caprylic/capric glycerides, D-alpha-tocopherol polyethylene glycol 1000 succinate, dimethyl sulfoxide, lipid, phospholipid, a cocrystal, hydrogenated soy phosphatidylcholine, distearoylphosphatidylglycerol, L-alpha-dimyristoylphosphatidylcholine, L-alpha-dimyristoylphosphatidylglycerol, N-methyl-2-pyrrolidone, methanol, isopropanol, hard fat, fat, lotion, milk, cream, petroleum jelly, polyethylene glycol 35 castor oil, ethylene glycol, propylene glycol, diethylene glycol, cyclodextrin, alpha-cyclodextrin, beta-cyclodextrin, hydroxypropyl-beta-cyclodextrin, sulfobutylether-beta-cyclodextrin, soy fatty acid, vinegar, glycerol, diethyl ether, fruit essence, orange essence, lemon essence, pineapple essence, gelatin, gelatin hydrolysate, xylitol, sorbitol, polyethylene glycol 40 stearate, polyethylene glycol 8 caprylic/capric glyceride, mannitol, and cellulose. The group may further comprise nicotinamide riboside, nicotinamide mononucleotide, nicotinamide adenine dinucleotide, calcium, calcium carbonate, ascorbic acid, vitamin C, vitamin D, vitamin D1, vitamin D2, vitamin D3, vitamin D4, vitamin D5, vitamin E, vitamin K, vitamin K1, and vitamin K2. The group may further comprise Kolliphor, Kolliphor CS 12, Kolliphor CS 20, Kolliphor CS S, Kolliphor CS A, Kolliphor CS L, Kolliphor TPGS, Kolliphor SLS, Kolliphor SLS Fine, Kolliphor RH 40, Kolliphor PS 20, Kolliphor PS 60, Kolliphor PS 80, Kolliphor HS 15, Kolliphor EL, Kolliphor P 188, Kolliphor P 407, Kolliphor P 237, Kolliphor P 338, Kolliphor P 188 micro, Kolliphor P 407 micro, Kollisolv, Kollisolv MCT 70, Kollisolv CAP, Kollisolv GTA, Kollisolv PG, Kollisolv PEG 300, Kollisolv PEG 400, Kollisolv PYR, Kollisolv P 124, Kolliphor EL, Cremophor, Cremophor RH 40, Cremophor RH 410, Cremophor RH 60, Labrafil, Labrafil M-1944CS, Labrafil M-2125CS, Softigen 767, Labrasol, Gellucire, Gellucire 44/14, Solutol, and Solutol HS 15.

5. The fisetin composition of claim 1 is a pharmaceutical composition, medical composition, cosmetic composition, lotion composition, cream composition, scientific composition, research composition, dental composition, oral composition, veterinary composition, ophthalmologic composition, dermatological composition, topical composition, alcohol composition, beverage composition, sauce composition, food composition, injectable composition, intravenous composition, atomized composition, or sprayed composition.

6. The fisetin composition of claim 1, wherein said fisetin is a synthesized fisetin, purified fisetin, natural fisetin, plant fisetin, tree fisetin, stem fisetin, leaf fisetin, fruit fisetin, vegetable fisetin, solvent extracted fisetin, or an enriched fisetin.

7. The fisetin composition of claim 1 is a tincture that is an ethanol tincture, vinegar tincture, diethyl ether tincture, glycerol tincture, or propylene glycol tincture.

8. The fisetin composition of claim 1 is a gel capsule.

9. The fisetin composition of claim 1 is an emulsion.

10. The fisetin composition of claim 1, wherein the fisetin composition further comprises a liposome, wherein said liposome comprises said fisetin and said second component.

11. The fisetin composition of claim 10, wherein said liposome comprises fisetin and water and ethanol and lecithin.

12. The fisetin composition of claim 1, wherein the fisetin composition further comprises a nanoparticle, wherein said nanoparticle comprises said fisetin and said second component.

13. The fisetin composition of claim 1, wherein the fisetin comprises at least a portion that is solubilized.

14. A kit capable of applying a fisetin composition, wherein the fisetin composition comprises: (a) fisetin; and (b) a second component selected from a group that comprises ethanol and water.

15. The kit of claim 14, said group further comprises oil, castor oil, coconut oil, sunflower oil, flaxseed oil, grape seed oil, sesame oil, avocado oil, almond oil, coconut oil, corn oil, cottonseed oil, rapeseed oil, canola oil, safflower oil, olive oil, palm oil, peanut oil, peppermint oil, sesame oil, hydrogenated soybean oil, soybean oil, medium chain triglyceride oil, long chain triglyceride oil, mineral oil, hydrogenated vegetable oil, vegetable oil, polyoxyl 35 castor oil, polyoxyl 40 hydrogenated castor oil, polyoxyl 60 hydrogenated castor oil, polyoxyl hydrogenated castor oil, polyethoxylated castor oil, and polyethylene glycol 40 hydrogenated castor oil. The group further comprise lecithin, beeswax, wax, d-alpha-tocopherol, oleic acid, monoglyceride, diglyceride, triglyceride, water, ethanol, alcohol, glycerin, polyethylene glycol, polyethylene glycol 400, polyethylene glycol 300, polyethylene glycol 1750, sodium dodecyl sulfate, sodium lauryl sulfate, polyethylene glycol 660 12-hydroxystearate, polyethylene glycol hydroxystearate, polyethylene glycol 300 caprylic/capric glycerides, polyoxylglyceride, propanediol, glycine, polyoxyl 8 stearate, polyoxyl 40 stearate, triglyceride, tocopherol, sorbitan monooleate, sorbitan monostearate, sorbitan monolaurate, sorbitan monopalmitate, sorbitan, polyoxyl40 stearate, poloxamer, poloxamer 188, poloxamer 407, poloxamer, polyethylene oxide, polysorbate 20, polysorbate 80, polysorbate, polyethylene glycol glycerides, polyethylene glycol 300 linoleic glycerides, polyethylene glycol 300 oleic glycerides, polyethylene glycol 300 caprylic/capric glycerides, polyethylene glycol 400 linoleic glycerides, polyethylene glycol 400 oleic glycerides, polyethylene glycol 400 caprylic/capric glycerides, D-alpha-tocopherol polyethylene glycol 1000 succinate, dimethyl sulfoxide, lipid, phospholipid, a cocrystal, hydrogenated soy phosphatidylcholine, distearoylphosphatidylglycerol, L-alpha-dimyristoylphosphatidylcholine, L-alpha-dimyristoylphosphatidylglycerol, N-methyl-2-pyrrolidone, methanol, isopropanol, hard fat, fat, lotion, milk, cream, petroleum jelly, polyethylene glycol 35 castor oil, ethylene glycol, propylene glycol, diethylene glycol, cyclodextrin, alpha-cyclodextrin, beta-cyclodextrin, hydroxypropyl-beta-cyclodextrin, sulfobutylether-beta-cyclodextrin, soy fatty acid, vinegar, glycerol, diethyl ether, fruit essence, orange essence, lemon essence, pineapple essence, gelatin, gelatin hydrolysate, xylitol, sorbitol, polyethylene glycol 40 stearate, polyethylene glycol 8 caprylic/capric glyceride, mannitol, and cellulose. The group further comprise nicotinamide riboside, nicotinamide mononucleotide, nicotinamide adenine dinucleotide, calcium, calcium carbonate, ascorbic acid, vitamin C, vitamin D, vitamin D1, vitamin D2, vitamin D3, vitamin D4, vitamin D5, vitamin E, vitamin K, vitamin K1, and vitamin K2. The group further comprise Kolliphor, Kolliphor CS 12, Kolliphor CS 20, Kolliphor CS S, Kolliphor CS A, Kolliphor CS L, Kolliphor TPGS, Kolliphor SLS, Kolliphor SLS Fine, Kolliphor RH 40, Kolliphor PS 20, Kolliphor PS 60, Kolliphor PS 80, Kolliphor HS 15, Kolliphor EL, Kolliphor P 188, Kolliphor P 407, Kolliphor P 237, Kolliphor P 338, Kolliphor P 188 micro, Kolliphor P 407 micro, Kollisolv, Kollisolv MCT 70, Kollisolv CAP, Kollisolv GTA, Kollisolv PG, Kollisolv PEG 300, Kollisolv PEG 400, Kollisolv PYR, Kollisolv P 124, Kolliphor EL, Cremophor, Cremophor RH 40, Cremophor RH 410, Cremophor RH 60, Labrafil, Labrafil M-1944CS, Labrafil M-2125CS, Softigen 767, Labrasol, Gellucire, Gellucire 44/14, Solutol, and Solutol HS 15.

16. The kit of claim 14, further comprises:
a container for mixing the fisetin and a second component; and
a label comprising a set of instructions for use.

17. The kit of claim 14, wherein the fisetin composition is a pharmaceutical composition, medical composition, cosmetic composition, lotion composition, cream composition, scientific composition, research composition, dental composition, oral composition, veterinary composition, ophthalmologic composition, alcohol composition, beverage composition, sauce composition, food composition, injectable composition, intravenous composition, atomized composition, or sprayed composition.

18. A fisetin composition comprising:
a first component selected from a first group that comprises fisetin; and
a second component selected from a second group that comprises polyethylene glycol.

19. The fisetin composition of claim 18, wherein the second group comprises water and polyethylene glycol.

20. The fisetin composition of claim 18, wherein a third component is selected from a third group that comprises a nanoparticle.
